(19) United States
(12) Patent Application Publication
(10) Pub. No.: US 2009/0292301 A1
(43) Pub. Date: Nov. 26, 2009

(54) Tissue Fastener, and Tissue Fastener System and method Employing the Same

(76) Inventor: Carl Hasselman, Oakmont, PA (US)

Correspondence Address: ECKERT SEAMANS CHERIN & MELLOTT, 600 GRANT STREET, 44TH FLOOR, PITTSBURGH, PA, 15219, US

(21) Appl. No.: 12/447,539

(22) PCT Filed: Oct. 30, 2007
(86) PCT No.: PCT/US2007/082945

§ 371 Date: Apr. 28, 2009

Related U.S. Application Data
(60) Provisional application No. 60/865,989, filed on Nov. 15, 2006.

(51) Int. Cl. A61B 17/08; A61L 17/00

(52) U.S. Cl. 606/151; 606/228

(57) ABSTRACT

A fastener (30,130) is provided for securing a soft tissue (4) to a bone (6) upon actuation of an actuating mechanism (16,116). The fastener (30,130) includes an elongated tubular shaft (32,132) having first and second opposing ends (34,36; 134,136), an aperture (38,138) extending from the second end (36,136) toward the first end (34,134), and a collar (40,140) disposed at or about the second end (36,136). The first end (34,134) of the elongated tubular shaft (32,132) is inserted through the soft tissue (4), through the exterior surface (10) of the cortex (8) and into the interior (12) of the bone (6). The collar (40,140) engages and secures the soft tissue (4). In response to being actuated by the actuating mechanism (16,116), a portion (34,134) of the elongated tubular shaft (32,132) deforms against the cortex (8) within the bone (6), thereby securing the soft tissue (4) between the collar (40,140) and the exterior surface (10) of the cortex (8) of the bone (6). A tissue fastener system (2,102) and an associated method of fastening soft tissue (4) to a bone (6) are also disclosed.

### CROSS-REFERENCE TO RELATED APPLICATION

This application claims the benefit of U.S. Provisional Patent Application Ser. No. 60/865,989, filed Nov. 15, 2006.

### BACKGROUND OF THE INVENTION

1. Field of the Invention

The invention relates generally to fasteners and, more particularly, to fasteners for securing soft tissues such as, for example, ligaments, tendons and fascia, to bones. The invention also relates to tissue fastener systems. The invention further relates to methods employing tissue fasteners.

2. Background Information

Soft tissues such as, for example, ligaments and tendons, are attached to bones throughout the body and can detach from the bone, for example, as a result of an injury. At other times, it is necessary to attach soft tissue to bone as part of a grafting procedure, for example, in order to resurface a damaged joint (e.g., without limitation, ankle; elbow; foot; hand; hip; knee; shoulder; wrist). In any event, it is desirable to position the soft tissue as accurately as possible with respect to the bone, and to ensure that the soft tissue is as secure as possible.

Bones generally comprise a hard outer structure, known as cortical bone or simply the cortex, and an inner cavity which contains relatively soft matter, known as cancellous matter. Prior proposals for attaching soft tissue to bone have involved a wide variety of fastening mechanisms including, for example, sutures, tacks and rivet-like mechanisms. Some of these mechanisms are designed to be inserted through the cortex into the cancellous matter, while others are designed to be anchored solely in the cortex. Although there has been some success using such mechanisms, significant room for improvement remains. Specifically, each of the above fastening mechanisms suffers from its own unique disadvantages. For instance, proper application of sutures is demanding on the individual performing the procedure to implement them, and sutures require incisions which are larger than desired. Anchor mechanisms, such as barbed tacks and nails, sometimes have poor fixation in the bone, particularly where the bone is relatively soft (i.e., osteoporotic bone). Known rivet-like mechanisms have also been plagued by losing fixation in the bone over time, particularly in relatively soft or osteoporotic bone.

Many of the fixation difficulties associated with known tissue-to-bone fastening mechanisms result, in large part, from the fact that many such mechanisms are primarily focused on fixation with respect to the cancellous matter of the bone. There is a need, therefore, for a fastening mechanism which is capable of effectively and reliably securing soft tissues to cortical bone. There is a further need for such a mechanism which is relatively simple in design and easy to employ as part of a minimally invasive medical procedure.

There is, therefore, room for improvement in fasteners for securing soft tissue to bone, and in associated methods employing such fasteners.

### SUMMARY OF THE INVENTION

These needs and others are met by embodiments of the invention, which are directed to a fastener, a fastener system, and an associated method for securing a soft tissue such as, for example, a ligament, a tendon or fascia, to a cortex of a bone.

As one aspect of the invention, a fastener is provided for securing a soft tissue to a bone upon actuation of an actuating mechanism. The bone has a cortex with an exterior surface, and an interior. The fastener comprises: an elongated tubular shaft including a first end, a second end disposed opposite and distal from the first end, and an aperture extending from the second end of the elongated tubular shaft toward the first end of the elongated tubular shaft, the aperture being structured to be engaged by the actuating mechanism; and a collar disposed at or about the second end of the elongated tubular shaft. The first end of the elongated tubular shaft is structured to be inserted through the soft tissue, through the exterior surface of the cortex and into the interior of the bone, and the collar of the fastener is structured to engage and secure the soft tissue. In response to being actuated by the actuating mechanism, a portion of the elongated tubular shaft of the fastener is structured to deform against the cortex within the bone, thereby securing the soft tissue between the collar of the fastener and the exterior surface of the cortex of the bone.

The collar may include a first side structured to face the soft tissue and a second side disposed opposite the first side. A plurality of protrusions may extend outwardly from the first side of the collar, wherein the protrusions are structured to engage the soft tissue. The protrusions may be a plurality of conically-shaped barbs extending outwardly from the first side of the collar. The fastener may be made from a biocompatible material.

As another aspect of the invention, a tissue fastener system is provided for securing a soft tissue to a bone. The bone has a cortex with an exterior surface, and an interior. The tissue fastener system comprises: an actuating mechanism; and a fastener comprising: an elongated tubular shaft including a first end, a second end disposed opposite and distal from the first end, and an aperture extending from the second end of the elongated tubular shaft toward the first end of the elongated tubular shaft, the aperture receiving the actuating mechanism, and a collar disposed at or about the second end of the elongated tubular shaft of the fastener. The first end of the elongated tubular shaft of the fastener is structured to be inserted through the soft tissue, through the exterior surface of the cortex and into the interior of the bone, and the collar of the fastener is structured to engage and secure the soft tissue. In response to being actuated by the actuating mechanism, a portion of the elongated tubular shaft of the fastener is structured to deform against the cortex within the bone, thereby securing the soft tissue between the collar and the exterior surface of the cortex of the bone.

The fastener may further include at least one suture, and the collar may include a first side structured to face the soft tissue, a second side disposed opposite the first side of the collar, and a number of suture holes extending from the first side of the collar to the second side of the collar. The at least one suture may extend through a corresponding one of the number of suture holes, and the at least one suture may be structured to suture the soft tissue to the fastener.

The actuating mechanism may comprise a component including first end, a second end disposed opposite and distal from the first end of the component, and an elongated shaft extending between the first end and the second end. The first end of the component may include a trocar structured to facilitate insertion of the fastener into the bone. The component may further include a plurality of threaded portions, and the aperture of the elongated tubular shaft of the fastener may include a plurality of threads wherein, when the component is inserted into the aperture of the elongated tubular shaft, the threaded portions of the component engage the threads of the aperture of the elongated tubular shaft. The component may be a drill bit, wherein the actuating mechanism further comprises a drill for actuating the drill bit. The first end of the drill bit may be structured to be inserted into the aperture of the elongated tubular shaft of the fastener, and the second end of the drill bit may be structured to be coupled to the drill wherein, when the drill actuates the drill bit within the aperture of the elongated tubular shaft of the fastener, the drill bit is structured to outwardly expand at least a portion of the fastener toward engagement with the bone. Alternatively, the component of the actuating mechanism may be a screw wherein, when the screw is actuated within the aperture of the elongated tubular shaft of the fastener, the screw is structured to outwardly expand at least a portion of the fastener toward engagement with the bone.

As another aspect of the invention, a method of employing a tissue fastener system to fasten a soft tissue to a bone is provided. The tissue fastener system includes an actuating mechanism and a fastener. The bone has a cortex with an exterior surface, and an interior. The fastener includes an elongated tubular shaft with a first end, a second end disposed opposite and distal from the first end and including a collar, and an aperture extending from the second end of the elongated tubular shaft toward the first end of the elongated tubular shaft. The aperture receives the actuating mechanism. The method comprises: positioning the soft tissue in a desired location with respect to the exterior surface of the cortex of the bone; inserting the first end of the elongated tubular shaft of the fastener through the soft tissue, through the exterior surface of the cortex and into the bone until the first end of the elongated tubular shaft of the fastener is disposed within the interior of the bone; and actuating the actuating mechanism within the aperture of the elongated tubular shaft of the fastener, in order to deform at least a portion of the elongated tubular shaft of the fastener against the cortex within the bone, thereby securing the soft tissue between the collar of the fastener and the exterior surface of the cortex of the bone.

### BRIEF DESCRIPTION OF THE DRAWINGS

A full understanding of the invention can be gained from the following description of the preferred embodiments when read in conjunction with the accompanying drawings in which:

FIG. 1 is a side elevation view of a tissue fastener system for attaching soft tissue to bone in accordance with an embodiment of the invention;

FIG. 2 is a side elevation view of the actuator mechanism of the tissue fastener system of FIG. 1;

FIG. 3 is an isometric view of the tissue fastener of the tissue fastener system of FIG. 1;

FIG. 4 is a cross-sectional view taken along line 4-4 of FIG. 3;

FIGS. 5A, 5B and 5C are side elevation views of sequential steps of a method for attaching soft tissue to bone employing the tissue fastener system of FIG. 1, in accordance with another embodiment of the invention; and

FIGS. 6A and 6B are side elevation unactuated and actuated views, respectively, of a tissue fastener in accordance with another embodiment of the invention.

### DESCRIPTION OF THE PREFERRED EMBODIMENTS

For purposes of illustration, embodiments of the invention will be described as applied to a fastener for attaching a ligament to a bone, although it will become apparent that they could also be applied to attach any suitable soft tissue (e.g., without limitation, tendons; fascia) other than ligaments, to bone.

Directional phrases used herein, such as, for example, front, back, top, bottom, upper, lower, interior, exterior and derivatives thereof, relate to the orientation of the elements shown in the drawings and are not limiting upon the claims unless expressly recited therein.

As employed herein, the term “soft tissue” refers to any known or suitable human, animal or artificial tissue suitable for attachment to bone in the body, for example, in order to repair or reconstruct a joint, and expressly includes, without limitation, “allograft” which comprises soft tissue from a separate human source such as, for example, a living human donor or a cadaver, and “xenograft” which comprises soft tissue from an animal source.

As employed herein, the term “bone” refers to any known or suitable human, animal or artificial structure suitable for use in the body as a component of the skeleton.

As employed herein, the term “bioabsorbable” refers to any known or suitable material which may be safely employed within the body of a human or animal, and which has a tendency to degrade and be absorbed by the body over time when employed in the body, and expressly includes, without limitation, plylactic acid (PLA) and polyglycolic acid (PGA).

As employed herein, the term “biocompatible” refers to any known or suitable material which may be safely employed within the body of a human or animal, and which does not necessarily degrade over time, but rather is compatible for use within the body for an extended period of time (e.g., indefinitely) without degrading, and expressly includes, without limitation, plastics and metals which are suitable for safe use in the body.

As employed herein, the term “trocar” is used in accordance with its conventional meaning to refer to a sharp-pointed surgical instrument. The trocar may be fitted with a cannula and used, for example and without limitation, to insert the cannula into a body cavity. The trocar may also be used, for example, to facilitate insertion of a tissue fastener in accordance with embodiments of the invention, into structures of the body such as, for example and without limitation, ligaments, tendons, fascia and bones.

As employed herein, the term “fascia” is used in accordance with its conventional meaning to refer to a connective tissue covering or binding body structures (e.g., without limitation, muscles).

As employed herein, the statement that two or more parts are “coupled” together shall mean that the parts are joined together either directly or joined through one or more intermediate parts.

As employed herein, the term “number” refers to the quantity one or an integer greater than one (i.e., a plurality).

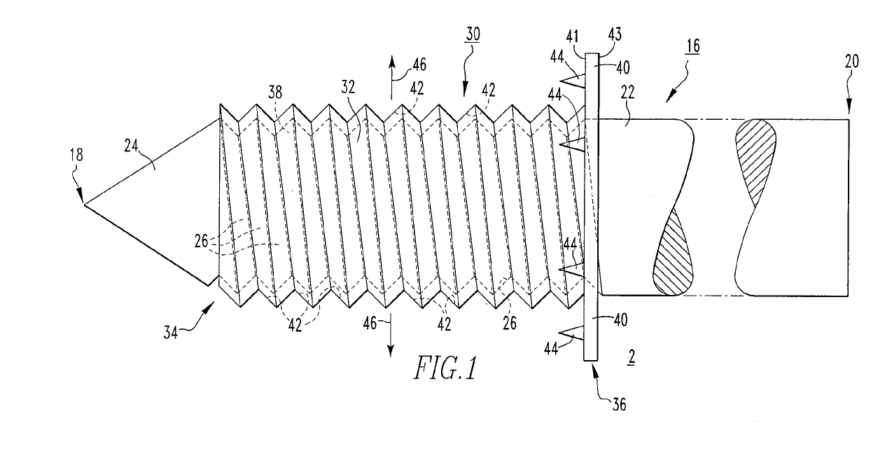

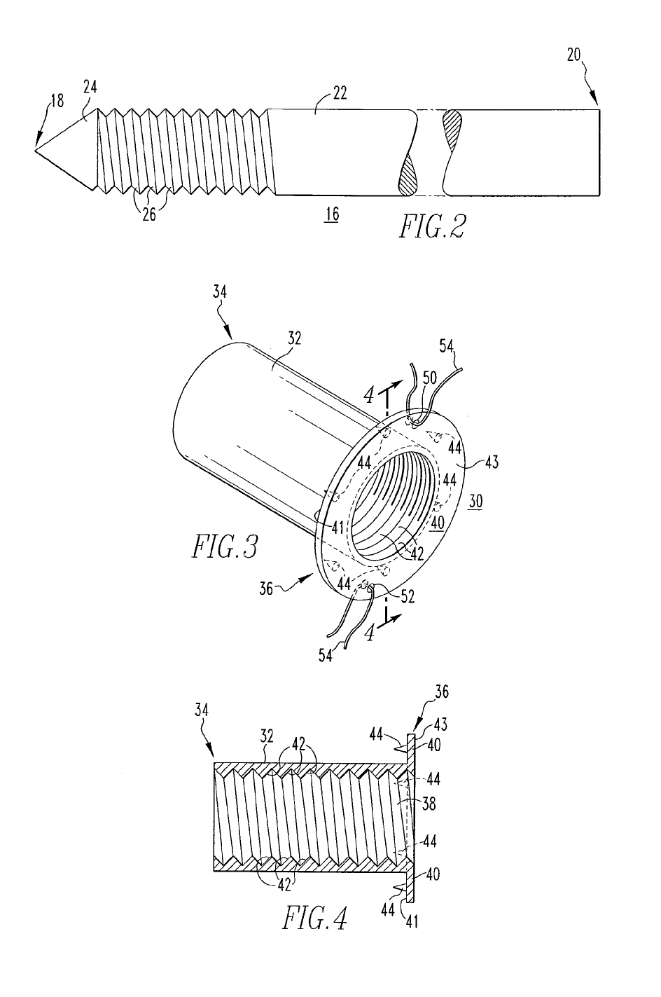

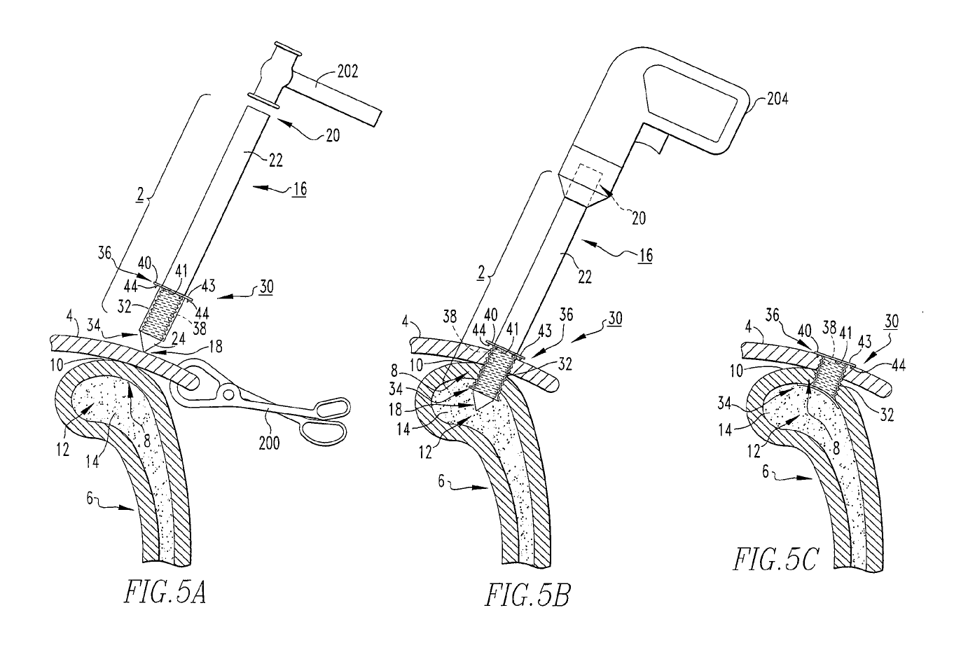

FIG. 1 shows a tissue fastener system 2 for attaching soft tissues 4 (e.g., without limitation, ligaments; tendons; fascia) (FIGS. 5A-5C) to bones 6 (e.g., without limitation, clavicle; scapula; humerus; radius; ulna; naicular; femur; patella; tibia; fibula; talus; calcaneus; midfoot bones) (FIGS. 5A-5C), in accordance with embodiments of the invention. The tissue fastener system 2 includes an actuating mechanism 16 (best shown in FIG. 2) and a fastener 30 (best shown in FIGS. 3 and 4).

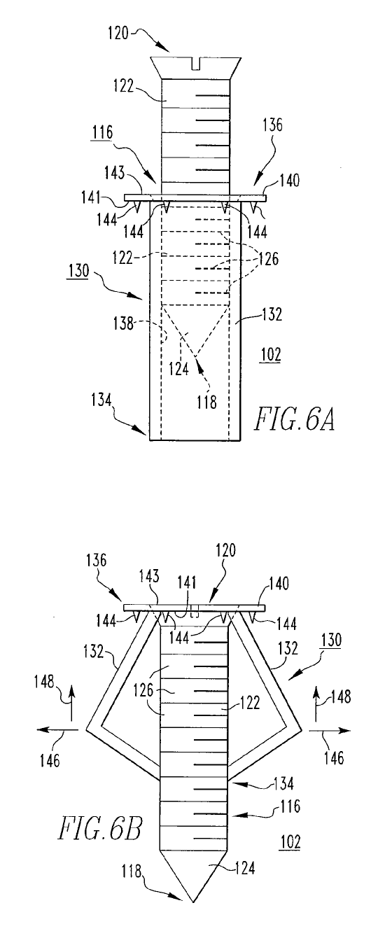

As shown in FIGS. 1 and 2, the example actuating mechanism 16 generally comprises a component (e.g., bit) for a drill, such as the hand drill 204 shown in FIG. 5B. More specifically, the actuating mechanism 16 includes a first end 18, a second end 20 and an elongated shaft 22 (shown in partially sectioned view in FIG. 1) extending therebetween. The first end 18 includes a trocar 24 for facilitating insertion of the fastener 30 into the bone 6 (FIGS. 5A-5B). A plurality of threads 26 (shown in hidden line drawing in FIG. 1) are disposed proximate the first end 18 and are structured to actuate (e.g., without limitation, expand radially outwardly) the fastener 30, for example and without limitation, in the directions indicated by arrows 46 of FIG. 1. In this manner, the actuating mechanism 16 actuates the fastener 30 of the tissue fastener system 2 in order that the fastener 30 securely engages a portion of the bone 6 (FIGS. 5A-5C), as will be discussed hereinbelow. It will, however, be appreciated that the actuating mechanism 16 could have any known or suitable alternative configuration in order to suitably install the fastener 30. For example and without limitation, the elongated shaft 22 of the actuating mechanism 16 could comprise a plurality of threaded portions (not shown) each having a different thread size and/or pitch, and/or the threads (e.g., 26) of the actuating mechanism 16 could gradually increase in size (e.g., without limitation, radial diameter) (not shown) as they progress from proximate the first end 18 of the actuating mechanism 16 toward the second end 20 of the actuating mechanism 16. Additionally, as will be shown and described with respect to FIGS. 6A and 6B hereinbelow, the actuating mechanism 16 could alternatively comprise a number of pieces or sections. For example and without limitation, the actuating mechanism 16 could comprise a screw, such as the screw 116 of FIGS. 6A and 6B, and a suitable component (e.g., bit) for engaging and actuating the screw. As will be discussed, the screw (see, for example, screw 116 in FIGS. 6A and 6B) may be structured to remain within the fastener 30 (see, for example, screw 116 within fastener 130 in FIG. 6B) after being actuated.

The fastener 30, as shown in FIGS. 1, 3 and 4, includes an elongated tubular shaft 32 (shown in its deformed or actuated configuration in FIG. 1) having a first end 34 and a second end 36 disposed opposite and distal from the first end 34. An aperture 38 extends from the second end 36 of the elongated tubular shaft 32 toward the first end 34. In the example shown and described herein, the aperture 38 extends the entire distance of the elongated tubular shaft 32, from the first end 34 to the second end 36, thereby comprising a thru hole. The aperture 38 is structured to be engaged by the actuating mechanism 16 (FIGS. 1, 2, 5A and 5B), as previously discussed and, in the example of FIGS. 1, 3 and 4, the aperture 38 is threaded to include a plurality of threads 42 (shown in hidden line drawing in FIG. 1) structured to be engaged by the corresponding threads 26 (FIGS. 1 and 2) of the actuating mechanism 16 (FIGS. 1 and 2). It will, however, be appreciated that the aperture 38 need not necessarily be threaded. It could, for example, originally be smooth and be subsequently deformed upon actuation, for example, by threaded insertion of the actuating mechanism 16 (FIGS. 1 and 2) and the threads 26 (FIGS. 1 and 2) thereof. For example, the aperture 138 of fastener 130 described hereinbelow in connection with FIGS. 6A and 6B, does not contain threads prior to insertion and actuation of the actuating mechanism 116.

The fastener 30 also includes a collar 40 disposed at or about the second end 36 of the elongated tubular shaft 32. The collar 40 has first and second sides 41,43 (see also first and second sides 141,143 of collar 140 in FIGS. 6A and 6B). Preferably, the collar 40 includes a plurality of protrusions 44 which are suitably structured to engage and secure soft tissue 4 (FIGS. 5A-5C). In the example shown and described herein, the protrusions 44 (see also protrusions 144 in FIGS. 6A and 6B) are conically-shaped barbs which extend outwardly from the first side 41 of the collar 40 of the fastener 30, as shown. It will be appreciated that although the example fastener 30 includes six example barb protrusions 44 (best shown in FIG. 3) disposed generally equidistant with respect to one another about the fastener collar 40, any known or suitable alternative tissue engaging and securing mechanism (not shown) other than the example barb protrusions 44 could be employed in any known or suitable alternative number and configuration, without departing from the scope of the invention.

As shown in FIG. 3, the example collar 40 optionally includes a number of suture holes 50,52. Specifically, first and second suture holes 50,52 are provided for receiving sutures 54. Accordingly, the fastener 30 can be still further affixed in the desired location, for example, by suturing the fastener 30 to the surrounding tissue 4 (FIGS. 5A-5C). However, it will be appreciated that such suture holes (e.g., 50,52), or any other known or suitable attachment mechanism (not shown) for receiving sutures 54, could be employed in any suitable number and configuration with the fastener 30, or with fastener 130 discussed hereinbelow with respect to FIGS. 6A and 6B. It will also be appreciated that no such attachment mechanism (e.g., suture holes 50,52) is required.

Referring to FIGS. 5A-5C, a method of attaching soft tissue 4 to a bone 6 using the disclosed tissue fastener mechanism 2, will now be discussed. Specifically, as shown in FIG. 5A, in a first step of the method, soft tissue 4 such as, the example tendon which is shown, is positioned in the desired location with respect to the exterior surface 10 of the cortex 8 of the bone 6. To facilitate such positioning of the soft tissue 4, any known or suitable instrument such as, for example and without limitation, the forceps 200 shown in FIG. 5A, can be employed. The first end 34 (FIGS. 5A and 5B) of the elongated tubular shaft 32 (FIGS. 5A and 5B) of the fastener 30 is then inserted through the soft tissue 4, through the exterior surface 10 of the cortex 8, and into the bone 6 until the first end 34 of the elongated tubular shaft 32 of fastener 30 is disposed within the interior 12 of the bone 6, as shown in FIG. 5B. The aforementioned trocar 24 of the actuating mechanism 16 serves to facilitate this process by penetrating the relatively hard cortex 8. Specifically, the fastener 30 can be initially threaded onto the first end 18 of the actuating mechanism 16 in order that the trocar 24 extends beyond the first end 34 of the fastener 30, as shown in FIG. 5A. A hammer 202 or any other known or suitable instrument can then be used, for example, to tap on the second end 20 of the elongated shaft 22 of actuating mechanism 16 until the trocar 24 penetrates the bone 6 and the fastener 30 is positioned in the desired location therein. When the first end 34 of the elongated tubular shaft 32 of the fastener 30 is disposed within the interior 12 of the bone 6, as shown in FIG. 5B, the aforementioned barbs 44 (FIG. 5A) of the fastener collar 40 engage the soft tissue 4 and help to hold it in place with respect to the exterior surface 10 of bone 6.

Next, a drill, such as the hand drill 204 shown in FIG. 5B, is employed to rotate or otherwise suitably manipulate the actuating mechanism 16, thereby actuating (e.g., without limitation, expanding radially outwardly) the elongated tubular shaft 32 of the fastener 30. In the example of 5B, the second end 20 of the elongated shaft 22 of actuating mechanism 16 is coupled to the hand drill 204, and rotated thereby. Movement of the actuating mechanism 16 within the aperture 38 of the elongated tubular shaft 32 of fastener 30 expands the fastener 30, at least at the first end 34 of the elongated tubular shaft 32 thereof. More specifically, as shown, at least a portion (e.g., without limitation, first end 34) of the elongated tubular shaft 32 deforms against the cortex 8 within the bone 6. In this manner, the soft tissue 4 is secured between the collar 40 of the fastener 30 and the exterior surface 10 of the cortex 8 of the bone 6, as best shown in FIG. 5C.

FIGS. 6A and 6B illustrate a non-limiting example of an alternative tissue fastener system 102, and actuating mechanism 116 and fastener 130 therefor. Specifically, the actuating mechanism 116 in the example of FIGS. 6A and 6B comprises any known or suitable traditional fastener, such as the threaded screw 116 which is shown. It will be appreciated that the screw 116 and, for that matter, all of the components of the tissue fastener system 102, and tissue fastener system 2 previously discussed in connection with FIGS. 1-4 and 5A-5C, are preferably made from a suitable biocompatible material and more preferably, are made from a suitable bioabsorbable material such as, for example and without limitation, plylactic acid (PLA) or polyglycolic acid (PGA).

The screw 116 includes a first end 118, a second end 120 and an elongated shaft 122 extending therebetween. In the example of FIGS. 6A and 6B, the shaft 122 includes a plurality of threads 126. The fastener 130 includes a first end 134 and a second end 136 having a collar 140. Prior to the fastener 130 being actuated by actuating mechanism 116, as shown in FIG. 6B, the fastener 130 has an elongated tubular shaft 132 which extends between the first and second ends 134,136, as shown in FIG. 6A. An aperture 138 extends from the first end 134 to the second end 136 and is structured to be engaged by the screw 116. As previously discussed, it will be appreciated that the aperture 138 can be, but need not necessarily be, threaded (not shown). The tissue fastener mechanism 102 is employed in much the same manner as tissue fastener mechanism 2 previously described with respect to FIGS. 5A-5C. Specifically, insertion of the fastener 130 into the bone 6 (FIGS. 5A-5C) is facilitated by the trocar 124 of the screw 116. It will, however, be appreciated that the process of inserting the fastener 130, or fastener 30 of (FIGS. 1, 3, 4 and 5A-5C, could further be facilitated by first drilling a pilot hole (not shown) in the bone 6 (FIGS. 5A-5C).

Once inserted, the fastener 130 and, in particular, the elongated tubular shaft 132 of the fastener 130, is actuated by turning the screw 116 within the aperture 138 (FIG. 6A) of the fastener 130. It will be appreciated that the screw 116 could be turned using any known or suitable tool or mechanism such as, for example and without limitation, the hand drill 204 (having a suitable bit (not shown)) of FIG. 5B. It will also be appreciated that the actuating mechanism 16 previously discussed in connection with FIGS. 1, 2, 5A and 5B could be employed instead of screw 116 to actuate the fastener 130. Such turning of the screw 116 results in a portion of the elongated tubular shaft 132 of the fastener 130 deforming outwardly (from the perspective of FIG. 6B) in the direction of arrows 146, and upwardly (from the perspective of FIG. 6B) in the direction of arrows 148, as shown. In this manner, such portion (e.g., 132) of the fastener 130 is structured to deform against the cortex 8 (FIGS. 5A-5C) within the bone 6 (FIGS. 5A-5C) in much the same manner as the portion (e.g., elongated tubular shaft 32) previously described with respect to fastener 30 in the example of FIGS. 5A-5C. It will be appreciated that after actuation of the fastener 130 has been completed, the soft tissue 4 will be engaged and secured by barbs 144 of the collar 140 of the fastener 130, between the first side 141 of the collar 140 and the exterior surface 10 (FIGS. 5A-5C) of the cortex 8 (FIGS. 5A-5C) of bone 6 (FIGS. 5A-5C) in much the same way that fastener 30 secures soft tissue 4 in the example of FIG. 5C. It will also be appreciated that the screw 116 or other suitable actuating mechanism (not shown) could then be removed or remain permanently in place.

Accordingly, the disclosed tissue fastener system 2,102, and actuating mechanisms 16,116 and fasteners 30,130 therefor provide a means for accurately, reliably and effectively attaching soft tissue 4 to bone 6 which is relatively simple in construction and easy to implement, and which is cost effective to make and use.

While specific embodiments of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of the invention which is to be given the full breadth of the claims appended and any and all equivalents thereof.

What is claimed is:

1. A fastener for securing a soft tissue to a bone upon actuation of an actuating mechanism, said bone having a cortex with an exterior surface, and an interior, said fastener comprising:
an elongated tubular shaft including a first end, a second end disposed opposite and distal from the first end, and an aperture extending from the second end of said elongated tubular shaft toward the first end of said elongated tubular shaft, said aperture being structured to be engaged by said actuating mechanism; and
a collar disposed at or about the second end of said elongated tubular shaft,
wherein the first end of said elongated tubular shaft is structured to be inserted through said soft tissue, through the exterior surface of said cortex and into the interior of said bone,
wherein said collar of said fastener is structured to engage and secure said soft tissue, and
wherein, in response to being actuated by said actuating mechanism, a portion of said elongated tubular shaft of said fastener is structured to deform against said cortex within said bone, thereby securing said soft tissue between said collar of said fastener and said exterior surface of said cortex of said bone.

2. The fastener of claim 1 wherein said collar comprises a first side structured to face said soft tissue and a second side disposed opposite the first side of said collar; wherein the first side of said collar comprises a plurality of protrusions extending outwardly from the first side of said collar; and wherein said protrusions are structured to engage said soft tissue.

3. The fastener of claim 2 wherein said protrusions are a plurality of conically-shaped barbs extending outwardly from the first side of said collar.

4. The fastener of claim 2 wherein said collar further comprises a number of suture holes; and wherein said number of suture holes extend from the first side of said collar to the second side of said collar.

5. The fastener of claim 1 wherein said fastener is made from a biocompatible material.

6. The fastener of claim 1 wherein said aperture comprises a thru hole extending from the first end of said elongated tubular shaft to the second end of said elongated tubular shaft.

7. The fastener of claim 1 wherein said actuating mechanism includes a plurality of threaded portions; wherein said aperture includes a plurality of threads; and wherein said threads of said aperture are structured to be engaged by said threaded portions of said actuating mechanism.

8. A tissue fastener system for securing a soft tissue to a bone, said bone having a cortex with an exterior surface, and an interior, said tissue fastener system comprising:
an actuating mechanism; and
a fastener comprising:
an elongated tubular shaft including a first end, a second end disposed opposite and distal from the first end, and an aperture extending from the second end of said elongated tubular shaft toward the first end of said elongated tubular shaft, said aperture receiving said actuating mechanism, and
a collar disposed at or about the second end of said elongated tubular shaft of said fastener,
wherein the first end of said elongated tubular shaft of said fastener is structured to be inserted through said soft tissue, through the exterior surface of said cortex and into the interior of said bone,
wherein said collar of said fastener is structured to engage and secure said soft tissue, and
wherein, in response to being actuated by said actuating mechanism, a portion of said elongated tubular shaft of said fastener is structured to deform against said cortex within said bone, thereby securing said soft tissue between said collar and said exterior surface of said cortex of said bone.

9. The tissue fastener system of claim 8 wherein said collar comprises a first side structured to face said soft tissue and a second side disposed opposite the first side of said collar; wherein the first side of said collar comprises a plurality of protrusions extending outwardly from the first side of said collar; and wherein said protrusions are structured to engage said soft tissue.

10. The tissue fastener system of claim 9 wherein said protrusions are a plurality of conically-shaped barbs extending outwardly from the first side of said collar.

11. The tissue fastener system of claim 8 wherein said fastener further comprises at least one suture; wherein said collar comprises a first side structured to face said soft tissue, a second side disposed opposite the first side of said collar, and a number of suture holes extending from the first side of said collar to the second side of said collar; wherein said at least one suture extends through a corresponding one of said number of suture holes; and wherein said at least one suture is structured to suture said soft tissue to said fastener.

12. The tissue fastener system of claim 8 wherein said actuating mechanism comprises a component including first end, a second end disposed opposite and distal from the first end of said component, and an elongated shaft extending between the first end of said component and the second end of said component; and wherein the first end of said component includes a trocar structured to facilitate insertion of said fastener into said bone.

13. The tissue fastener system of claim 12 wherein said component of said actuating mechanism further includes a plurality of threaded portions; wherein said aperture of said elongated tubular shaft of said fastener includes a plurality of threads;
and wherein, when said component is inserted into said aperture of said elongated tubular shaft, said threaded portions of said component engage said threads of said aperture of said elongated tubular shaft.

14. The tissue fastener system of claim 12 wherein said component of said actuating mechanism is a drill bit including a first end and a second end; wherein said actuating mechanism further comprises a drill for actuating said drill bit; wherein the first end of said drill bit is structured to be inserted into said aperture of said elongated tubular shaft of said fastener; wherein the second end of said drill bit is structured to be coupled to said drill; and wherein, when said drill actuates said drill bit within said aperture of said elongated tubular shaft of said fastener, said drill bit is structured to outwardly expand at least a portion of said fastener toward engagement with said bone.

15. The tissue fastener system of claim 12 wherein said component of said actuating mechanism is a screw; and wherein, when said screw is actuated within said aperture of said elongated tubular shaft of said fastener, said screw is structured to outwardly expand at least a portion of said fastener toward engagement with said bone.

16. A method of employing a tissue fastener system to fasten a soft tissue to a bone, said tissue fastener system including an actuating mechanism and a fastener, said bone having a cortex with an exterior surface, and an interior, said fastener including an elongated tubular shaft with a first end, a second end disposed opposite and distal from the first end and including a collar, and an aperture extending from the second end of said elongated tubular shaft toward the first end of said elongated tubular shaft, said aperture receiving said actuating mechanism, said method comprising:
positioning said soft tissue in a desired location with respect to said exterior surface of said cortex of said bone;
inserting the first end of said elongated tubular shaft of said fastener through said soft tissue, through the exterior surface of said cortex and into said bone until the first end of said elongated tubular shaft of said fastener is disposed within the interior of said bone; and
actuating said actuating mechanism within said aperture of said elongated tubular shaft of said fastener, in order to deform at least a portion of said elongated tubular shaft of said fastener against said cortex within said bone, thereby securing said soft tissue between said collar of said fastener and said exterior surface of said cortex of said bone.

17. The method of claim 16, further comprising:
engaging said soft tissue with a pair of forceps, and
employing said pair of forceps to position said soft tissue in said desired location with respect to said exterior surface of said cortex of said bone.

18. The method of claim 16, further comprising inserting said fastener until a plurality of protrusions of said collar engage said soft tissue.

19. The method of claim 16, further comprising:
inserting at least one suture through a corresponding suture hole in said collar of said fastener, and
suturing said soft tissue to said fastener.

20. The method of claim 16, further comprising:
inserting a first end of a component of said actuating mechanism into said aperture of said elongated tubular shaft of said fastener, and
actuating said component of said actuating mechanism at or about a second end of said component, in order to outwardly expand at least a portion of said fastener toward engagement with said bone.

21. The method of claim 20, further comprising:
prior to actuating said component of said actuating mechanism, hammering the second end of said component of said actuating mechanism, and
penetrating said bone with a trocar disposed on the first end of said component of said actuating mechanism.

22. The method of claim 20 wherein said component of said actuating mechanism further includes a plurality of threaded portions; wherein said aperture of said elongated tubular shaft of said fastener includes a plurality of threads; and wherein said method further comprises:
engaging said threads of said aperture of said elongated tubular shaft of said fastener with said threaded portions of said component of said actuating mechanism, and
turning said component of said actuating mechanism in order to outwardly expand at least a portion of said elongated tubular shaft of said fastener toward engagement with said bone.

23. The method of claim 20 wherein said component of said actuating mechanism is a drill bit having a first end and a second end; and wherein said method further comprises:
inserting the first end of said drill bit into said aperture of said elongated tubular shaft of said fastener,
coupling the second end of said drill bit to a drill, and
actuating said drill in order to turn said drill bit within said aperture of said elongated tubular shaft of said fastener, thereby outwardly expanding at least a portion of said fastener toward engagement with said bone.

24. The method of claim 20 wherein said component of said actuating mechanism is a screw including a first end and a second end; and wherein the method further comprises:
inserting the first end of said screw into said aperture of said elongated tubular shaft of said fastener, and
turning said screw within said aperture of said elongated tubular shaft in order to outwardly expand at least a portion of said fastener toward engagement with said bone.
